(19) United States
(12) Patent Application Publication
(10) Pub. No.: US 2022/0193305 A1
(43) Pub. Date: Jun. 23, 2022

(54) CROSSLINKED SILK FIBROIN-BASED COMPOSITIONS, AND METHODS OF MAKING AND USING THE SAME

(71) Applicant: Trustees of Tufts College, Medford, MA (US)

(72) Inventors: Nicole Raia, Maynard, MA (US); David L. Kaplan, Medford, MA (US); Chiara E. Ghezzi, Lexington, MA (US)

(21) Appl. No.: 17/593,965

(22) PCT Filed: Mar. 30, 2020
(86) PCT No.: PCT/US2020/025790

§ 371 Date: Sep. 29, 2021

Related U.S. Application Data
(60) Provisional application No. 62/826,770, filed on Mar. 29, 2019.

(51) Int. Cl. A61L 27/26; A61L 27/52

(52) U.S. Cl. CPC A61L 27/26; A61L 2430/16; A61L 2400/06; A61L 27/52

(57) ABSTRACT

The present disclosure relates to biocompatible injectable compositions. The provided compositions comprise or consist of silk fibroin, hyaluronic acid, horseradish peroxidase, hydrogen peroxide, and water. The injectable composition is tunable and may be adapted to have a gelation time from 3 minutes to 20 minutes, a storage modulus of 6 Pa to 4000 Pa, be injectable through a needle having a size from 32 G to 18 G, have optical transmittance from 75% to 95% for at least one wavelength from 400 nm to 700 nm, have a volume expansion from 5% to 400% relative to an original volume of the hydrogel after soaking in an aqueous solution for 12 hours, and/or have a hydrogel stability by maintaining at least 75% of the storage modulus and the optical transmittance of the hydrogel after 6 months in vivo. The present disclosure provides methods for making and using the same.

### CROSS-REFERENCE TO RELATED APPLICATIONS

This patent application claims the benefit of priority of U.S. Provisional Application Ser. No. 62/826,770 filed on Mar. 29, 2019, the contents of which are hereby incorporated by reference herein.

### STATEMENT REGARDING FEDERALLY FUNDED RESEARCH

This invention was made with government support under grant number EB002520 awarded by National Institutes of Health. The government has certain rights in the invention.

### BACKGROUND

The vitreous humor is a clear, hydrated, avascular, gel-like network consisting of about 80% of the entire ocular volume. It is located between the retina and the lens, filling the vitreous cavity. The vitreous is attached to the surrounding ocular tissues at the anterior retina, macula, and optic nerve disc. There are many roles for the vitreous humor, including maintaining ocular volume, supporting and protecting the retina, facilitating metabolite diffusion, and contributing to the light pathway providing proper vision. In addition, the vitreous functions as a barrier for surrounding ocular tissues and provides mechanical dampening to prevent tissue damage.

The vitreous is considered a connective tissue that is composed of GAGs (HA, chondroitin sulfate, and heparan sulfate) and a small percentage of proteins (albumin, immunoglobulin, collagen, fibrillin, opticin, fibronectin, transferrin). The vitreous is primarily acellular with only a few cell types present, generally on the periphery of the vitreous body, including fibroblasts, hyalocytes, and macrophages that facilitate matrix formation, maintenance, and degradation. The viscoelasticity and rheological properties of the vitreous are a result of the interaction between a network of unbranched collagen fibrils within a bulk HA matrix. Understanding vitreous mechanics is ongoing and mostly involves porcine vitreous humor as a model. Human vitreous, obtained post-mortem, exhibits a shear modulus between 2-38 Pa.

Complications with the vitreous can be due to aging or trauma, as well ocular diseases and disorders including diabetic retinopathy, age-related macular degeneration (AMD), chorioretinitis, high myopia, and cancer. These indications can cause vitreous degeneration or posterior vitreous detachment (PVD), retinal detachment, retinal tears, and macular holes, which can lead to visual impairment. Vitreoretinal interface changes are key contributors to the complications occurring in diabetic retinopathy, especially the proliferative stage of the disease. In severe cases, a vitrectomy is performed, which involves removal of the vitreous. This procedure is the 3rd most frequently performed ophthalmic surgery with 225,000 vitrectomies performed a year in the US. Upon removal, the vitreous must then be replaced to maintain ocular volume and promote retinal attachment.

Loss of vitreous leads to accelerated cataract formation and increase in the health care cost. Retinal detachment is regarded as one of the top causes of permanent vision loss, where annual incidence of rhegmatogenous retinal detachment (RRD) has been reported to be 5.4-18.2 per 100,000 people with a high peak incidence of 52.5 per 100,000 people between 55 and 59 years of age. Retinal detachment is caused by fluid leaking behind the retinal pigment epithelium. Also, retinal tear induced by posterior vitreous detachment can lead to retinal detachment.

Currently, the most common “tamponading” agents used for the treatment of retinal detachment are silicone oil and gases (air, sulfur hexafluoride, or perfluoropropane). However, these substitutes suffer from disadvantages and limitations. Silicone oil has to be removed via another trip to the operating room and has a long list of potential complications, such as keratopathy, cataract, glaucoma, proliferative vitreoretinopathy, and corneal decompensation. Gases avoid some of the limitations of silicone oil, but limit vision for several weeks post-operative and require specialized positioning of the patient as well as limiting travel. Other adverse effects of gas temponades include cataract formation and corneal endothelial changes. Thus, a vitreous substitute with physiological properties closer to the normal vitreous would be more desirable than currently available “tamponading” agents.

Hydrogels are attractive materials for vitreous humor substitutes due to their viscoelasticity, hydrophilicity, and potential to form in situ, allowing ease of injection. Many hydrogels have been explored as vitreous humor substitutes, including synthetic hydrogels such as polyvinyl alcohol (PVA) and poly(l-vinyl-2-pyrrolidinone) (PVP). These polymers are advantageous for this application due to their extended retention time, rheological properties, and optical clarity, but they can also be toxic to ocular tissues, are usually non-injectable, or exhibit vitreous opacification.

Synthetic oligo-tetra-polyethylene glycol (PEG) hydrogels has been explored as a vitreous substitute. While these materials showed injectability, low osmotic pressure (<typical pressure in the eye ˜1 kPa), and low cytotoxicity, other key parameters (e.g., quantitative characterization of optical properties, mechanical properties to match human vitreous for the low-concentration polymer (4.0 g/L) used, limitations with a two-step processes for hydrogel formation, lack of information on kinetics of gel disintegration) were not addressed.

Hyaluronic acid (HA) and collagen, are biologically similar to native vitreous humor (which consists of up to 400 μg/cm3 of HA and 532 μg/cm3 of collagen), but have limited retention times in vivo due to relatively fast degradation in vivo. To improve retention time, HA has been crosslinked through UV and dihydrizide, however these materials still exhibit relatively short-term stability. HA has been combined with other polymers such as gellan, a microbial anionic polysaccharide, however due to the instability of the physical crosslinks, this combination was also only for short-term use. Table 1 shows examples of clinically available or experimental materials for vitreous substitutes, however there are currently no available materials that are ideal for vitreous substitution.

TABLE 1
Clinically available or experimental vitreous humor (VH) substitutes.

| | | Documented | | |
|---|---|---|---|---|
| Type of | Method of | durations in | Advantages for | Limitations for |
| Material | Preparation | VIVO | VH substitute | VH substitute |
| PVA | Crosslinked by | Obvious | Biocompatible (up to 3 mo), can be | uncrosslinked molecules induce |
| | gamma | degradation at | crosslinked without chemical agents, | inflammation, unknown long term |
| | irradiation | 6 mo | transparent, similar to native VH | effects, retinal vacuolations, cannot |
| | | | mechanics | form in situ, degradable, increased |
| | | | | inflammation and opacification, |
| | | | | requires high concentration, requires |
| | | | | larger needle to inject without fracture |
| pHEMA | Crosslinking with | >12 mo | Non degradable, inert transparent, | Non injectable, cannot form in situ, |
| | ethylene glycol | | hydrophilic, biocompatible | unknown mechanics, monomers are |
| | | | | toxic, low water content |
| Silicone Oil | Pure PDMS or | Typically | Good tamponade effect, similar | Emulsification, ocular toxicity, |
| | mixed F6H8 | removed after | viscosity and transparency as native | reduced retinal contact due to |
| | | 6 mo to avoid | VH, injectable, non-degradable | hydrophobicity, removal process can |
| | | adverse | | cause complications, can migrate, |
| | | effects | | hydrophobic |
| HA | Crosslinked via | Ranges from | Easily injected, unlikely to migrate, | Relatively rapid degradation |
| | BDDE or ADH | 2-5 mo | biocompatible, isovolemic degradation | |
| | | | (24, 25), can be removed easily by | |
| | | | injecting hyaluronidases | |
| Collagen | Proctase-treated | | Easy to use, readily available, | Inflammation, limited retinal |
| | bovine skin | Few weeks | biochemically similar to native VH | reattachment (poor surface tension), |
| | | | | opacification fracture upon injection, |
| | | | | rapid degradation |
| Oligo-tetra- | First: mixing with | 410 days | injectability, low osmotic pressure (< | No quantitative optical properties, |
| PEG | excess thiols or | | typical pressure in the eye ~1 kPa), | mechanical properties not matched to |
| | maleimides, | | low cytotoxicity | human vitreous for low-concentration |
| | second: mixing | | | (4.0 g/L) used, two-step processes for |
| | two types of | | | hydrogel formation, lack of |
| | clusters | | | information on kinetics of gel |
| | | | | disintegration |

*Commercially available for use in glaucoma surgery, vitreous humor supplementation is still experimental
Abbreviations: VH—vitreous humor; PVA—polyvinyl alcohol; pHEMA—polyhydroxyethylmethacrylate; PDMS—polydimethylsiloxane; F6H8—perfluorohexyloctane; HA—hyaluronic acid; BDDE—1,4-butanediol diglycidyl ether; ADH—adipic acid dihydrazide; PEG—polyethylene glycol

### SUMMARY

The present disclosure addresses the aforementioned drawbacks by providing crosslinked silk fibroin-based compositions, methods of making, and using the same. The provided crosslinked silk fibroin-based compositions are useful in a number of applications, including soft tissue supplementation, soft tissue augmentation, and polymeric networks that can be utilized as scaffolds for biomedical applications. Example applications include, but are not limited to, vitreous humor substitutes, mucous replacements, joint lubricity compositions, vocal cord augmentation, and biofilm related applications.

In some embodiments, the provided crosslinked silk fibroin-based compositions overcome one or more limitations of prior compositions, where the prior compositions (i) cannot form in situ gelation on injection, (ii) require large gauge needles for injection, (iii) induce inflammation or are toxic to biological tissue, (iv) are vulnerable to opacification over time, (v) are vulnerable to degradation over short time periods (i.e., do survive in vivo for greater than 1 month, greater than 3 months, or greater than 6 months) resulting in loss of mechanical or optical properties for the desired application, and (vi) do not have mechanical or optical properties that match in vivo tissue mechanics.

In some embodiments, the provided crosslinked silk fibroin-based compositions overcome one or more limitations of the prior compositions by (i) having gelation times suitable for in situ gelation (e.g., from 3 to 20 minutes or 10 to 15 minutes), (ii) having mechanical properties that are tunable to match the desired tissue type and application (e.g., storage modulus from 6 to 4000 Pa), (iii) are stable in vivo for at least 1 month, or at least 3 months, or at least 6 months, and are resistant to proteolytic degradation, (iv) are optically clear with visible light (e.g., 400 to 700 nm) transmission values from 75% to 95%, (v) are biocompatible, and (vi) have tunable swelling properties (e.g., may be tuned to expand on injection from 5% to 400% of the original volume over a duration, or may be tuned to have a reduction in volume on injection over a duration, the reduction ranging from 0% to 50% of the original volume over a duration), and (vii) are capable of being injected through small gauge needles (e.g., 32 G or less).

In some embodiments, provided herein is a biocompatible injectable composition. The biocompatible injectable composition comprises or consists of silk fibroin, hyaluronic acid, horseradish peroxidase, hydrogen peroxide, and water. In some embodiments, the injectable composition undergoes gelation from a non-gelated solution to a hydrogel upon mixing of the silk fibroin, hyaluronic acid, horseradish peroxidase, hydrogen peroxide, and water. In some embodiments, a polymeric ratio of silk fibroin and hyaluronic acid and a horseradish peroxidase concentration are adapted to provide a gelation time from 3 minutes to 20 minutes. In some embodiments, a silk fibroin concentration, a hyaluronic acid concentration, the polymeric ratio, and a hydrogen peroxide concentration are adapted to provide a storage modulus from 6 Pa to 4000 Pa. In some embodiments, the injectable composition is adapted to provide a viscosity of the non-gelated solution that is adequately low to allow injection via a needle having a size from 32 gauge to 18 gauge. In some embodiments, the injectable composition is adapted to provide an optical transmittance of the hydrogel from 75% to 95% for at least one wavelength of between 400 nm and 700 nm inclusive. In some embodiments, the injectable composition is adapted to provide a hydrogel swellability of the hydrogel including a volume expansion from 5% to 400% relative to an original volume of the hydrogel after soaking in an aqueous solution for 12 hours. In some embodiments, the injectable composition is adapted to provide a hydrogel stability by maintaining at least 75% of the storage modulus and optical transmittance of the hydrogel after 6 months in vivo.

In other embodiments, provided herein is a method for forming a biocompatible injectable composition. In some embodiments, the method includes contacting silk fibroin, hyaluronic acid, horseradish peroxidase, hydrogen peroxide, and water in a solution in amounts sufficient such that the solutions undergoes gelation to a hydrogel. In some embodiments, a polymeric ratio of the silk fibroin and the hyaluronic acid and a horseradish peroxidase concentration are adapted to provide a gelation time of from 3 minutes to 20 minutes. In some embodiments, a silk fibroin concentration, a hyaluronic acid concentration, the polymeric ratio, and a hydrogen peroxide concentration are adapted in the hydrogel to provide a storage modulus of from 6 Pa to 4000 Pa. In some embodiments, the injectable composition is adapted in the hydrogel to provide a viscosity of the non-gelated solution that is adequately low to allow injection via a needle having a size from 32 gauge to 18 gauge. In some embodiments, the injectable composition is adapted in the hydrogel to provide an optical transmittance of the hydrogel of between 75% and 95%, inclusive, for at least one wavelength of between 400 nm and 700 nm inclusive. In some embodiments, the injectable composition is adapted to provide a hydrogel swellability of the hydrogel including a volume expansion from 5% to 400%, inclusive, relative to an original volume of the hydrogel after soaking in an aqueous solution for 12 hours. In some embodiments, the injectable composition is adapted to provide a hydrogel stability by maintaining at least 75% of the storage modulus and the optical transmittance of the hydrogel after 6 months in vivo.

In some embodiments, provided herein is a kit comprising components for a biocompatible injectable composition. In some embodiments, the kit includes silk fibroin, hyaluronic acid, horseradish peroxidase, hydrogen peroxide, and water. In some embodiments, the silk fibroin, the hyaluronic acid, the horseradish peroxidase, the hydrogen peroxide, and the water are separated to prevent gelation (i.e., the components may have individual compartments to separate the components, or the kit may include two compartments dividing the components to separate the horseradish peroxidase, the silk fibroin, and the hyaluronic acid.). In some embodiments, when the components in the kit are combined the injectable composition undergoes gelation from a non-gelated solution to a hydrogel upon mixing of the silk fibroin, the hyaluronic acid, the horseradish peroxidase, the hydrogen peroxide, and the water. In some embodiments, a polymeric ratio of silk fibroin and hyaluronic acid and a horseradish peroxidase concentration are adapted to provide a gelation time from 3 minutes to 20 minutes. In some embodiments, a silk fibroin concentration, a hyaluronic acid concentration, the polymeric ratio, and a hydrogen peroxide concentration are adapted to provide a storage modulus from 6 Pa to 4000 Pa. In some embodiments, the injectable composition is adapted to provide a viscosity of the non-gelated solution that is adequately low to allow injection via a needle having a size from 32 gauge to 18 gauge. In some embodiments, the injectable composition is adapted to provide an optical transmittance of the hydrogel from 75% to 95% for at least one wavelength of between 400 nm and 700 nm inclusive. In some embodiments, the injectable composition is adapted to provide a hydrogel swellability of the hydrogel including a volume expansion from 5% to 400% relative to an original volume of the hydrogel after soaking in an aqueous solution for 12 hours. In some embodiments, the injectable composition is adapted to provide a hydrogel stability by maintaining at least 75% of the storage modulus and optical transmittance of the hydrogel after 6 months in vivo.

### DESCRIPTION OF THE DRAWINGS

The foregoing and other objects, aspects, features, and advantages of the present disclosure will become more apparent and better understood by referring to the following description taken in conjunction with the accompanying figures in which:

FIGS. 1(A-C) are graphs illustrating shear storage modulus data of the crosslinked silk fibroin-based compositions having varying concentrations of silk fibroin (S), glycosaminoglycan (H), and crosslinking agents (low, medium, high) according to some embodiments of the present disclosure. The day 1 storage modulus (A) for each ratio was within a low (below 30 Pa), medium (30-110 Pa), and high (above 110 Pa) range depending on H₂O₂ concentration. Storage modulus between 1 day and 1 month is shown for each sample (B) with 50S/50H ratios at 1 month having an 4×, 2×, and 1.6× increase in modulus for low, med, and high H₂O₂ concentrations respectively. For the same H₂O₂ concentrations, the storage modulus at 1 day and 1 month shows a direct relationship between storage modulus and HA ratios (C). (*p≤0.05, n=3-4).

FIGS. 2(A-H) are graphs illustrating storage modulus data for strain sweeps at 1 Hz for silk fibroin-based compositions having varying concentrations of silk fibroin (S), glycosaminoglycan (H), and crosslinking agent (low, medium, high) according to some embodiments of the present disclosure. Representative data for strain sweeps at 1 Hz of hydrogels with different S/H ratios and H₂O₂ concentrations are shown for 1 day (A-D) and 1 month (E-H). All hydrogels exhibited a viscoelastic linear region at 3% strain.

FIGS. 3(A-H) are graphs illustrating storage modulus data for frequency sweeps at 3% strain for silk fibroin-based compositions having varying concentrations of silk fibroin (S), glycosaminoglycan (H), and crosslinking agent (low, medium, high) according to some embodiments of the present disclosure. Representative data for frequency sweeps at 3% strain of hydrogels with different S/H ratios and H2O2 concentrations are shown for 1 day (A-D) and 1 month (E-H). All hydrogels were frequency independent except for low H2O2 concentrations for 25S/75H, 10S/90H, and 0S/100H which showed a slight increase in shear storage modulus at higher frequencies.

FIGS. 4(A-H) are graphs illustrating optical transparency as percentage of light transmitted through the silk fibroin-based compositions having varying concentrations of silk fibroin (S) and glycosaminoglycan (H) according to some embodiments of the present disclosure. Optical transparency is reported as percentage of light transmitted at day 1 (A-D) and 1 month (E-H) for each of the polymeric ratios. Statistical differences from the HBSS control at 500 nm are shown ([low], [med], [high] p≤0.05, n=5).

FIGS. 5(A-F) are graphs illustrating volumetric changes over time for the crosslinked silk fibroin-based compositions according to some embodiments of the present disclosure. Volumes are reported for hydrogels over a 1-month period (A-D). The time at which the volume no longer changes is shown in (E) where hydrogels with increased HA concentration and higher H₂O₂ concentration equilibrated faster. When comparing between ratios of the same H₂O₂ concentration (F), the 50S/50H hydrogels had a lower normalized volume than all other ratios (+0.05, *p≤0.05 compared to initial volume, n=5).

FIGS. 6(A-F) are graphs and images illustrating characterization data of the crosslinked silk fibroin-based compositions according to some embodiments of the present disclosure. (A) Schematic showing the crosslinking of silk and glycosaminoglycan (e.g, HA) into a composite hydrogel using crosslinking agents (e.g., HRP and H2O2). (B) Silk-HA hydrogels were visually clear and injectable. (C) The compressive moduli of the hydrogels, expressed on a log scale, were dependent on HA concentration where increasing concentration reduced the modulus increase after 1 month. (n=5, +p≤0.05 compared to 0%, *p≤0.05, ***p<0.001. Statistical analysis was performed after log transformation). (D) The ratio of ß-sheet to random coil conformation was calculated from the FTIR data by dividing the average of peak absorbance at 1620-1625 and 1640-1650 cm-1 showing that increasing HA concentration reduced the ratio over time. (n=5, *p≤0.05 and ***p≤0.001 compared to week 0). (E) Degradation was determined by calculating the fraction of initial mass after exposure in an enzymatic cocktail consisting of 1 U/mL and 0.001 U/mL of hyalruonidase (type I-S from bovine testes) and protease XIV (type XIV from Streptomyces griseus). A direct relationship between degradation rate and HA concentration was observed when increasing the HA concentration above 5%, the degradation rate significantly increased by day 8 (n=4, **p<0.01, ***p<0.001). (F) Live/dead staining of 2D cultures of hMSCs at day 3 showed that both silk and silk-HA (10% HA) hydrogels maintained cell viability similar to that of tissue culture plastic controls whereas, HA only hydrogels showed limited viable cells due to its low mechanical strength and anti-adhesive properties. (n=3, scale bar=100 μm, live cells and dead cells were stained green and red, respectively)

FIG. 7 is a schematic illustration of a crosslinking agent that facilitates crosslinking of the silk fibroin and the glycosaminoglycan via click chemistry according to some embodiments of the present disclosure.

FIGS. 8(A-C) are images of in vivo responses to crosslinked silk fibroin-based compositions according to some embodiments of the present disclosure. (A) crosslinked silk fibroin-based compositions injected into the cervix of rats and in vivo response 3 days post injection and post-partum determined by H&E. Arrows indicate silk gel; scale bar=200 μm. (B) Protein assay for inflammatory cytokines, IL-6 and IL-6; silk injections showed tissue cytokine levels either equivalent to saline or intermediate between saline and cerclage, with cerlcalge being highest (*p<0.05 vs saline; #p<0.05 vs cerclage). (C) Biocompatiblility of silk/HA particle suspensions assessed in a rat subcutaneous model. Histological examination showed cellular infiltration around the vicinity of the silk particles (yellow arrows) predominantly macrophages within the tissue ingrowth. Regions of HA (blue arrows) were cell occlusive and collapse upon histological processing (scale bar=0.125 mm).

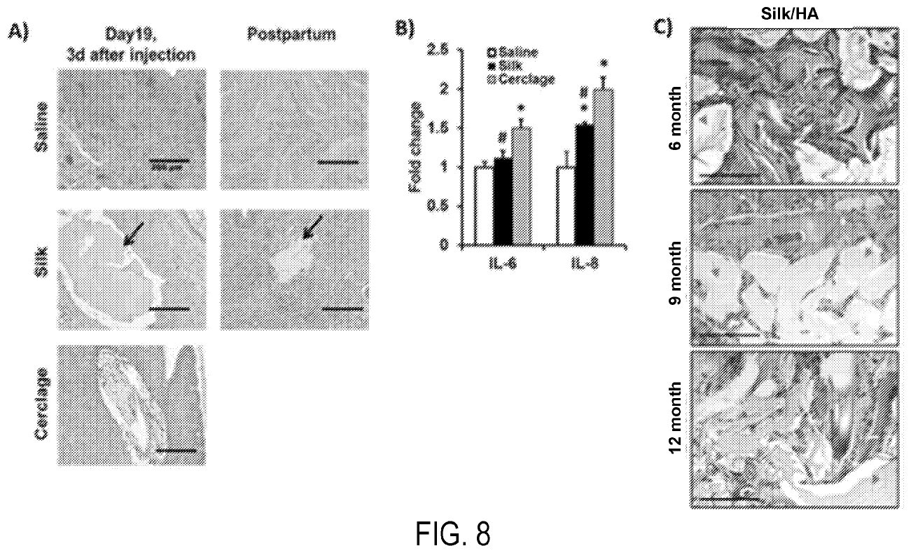

FIG. 9 is an in vivo assessment of a crosslinked silk fibroin-based composition in mouse vitreous to assess toxicity and compatibility according to some embodiments of the present disclosure. Two uL of silk hydrogel injected into the vitreous cavity of mice under approval of the MEEI animal protocols (N=3). Ocular examination prior to enucleation was normal. No apoptosis was detected by TUNEL and no Muller glia or astrocyte activation was noted. Graph: p<0.05.

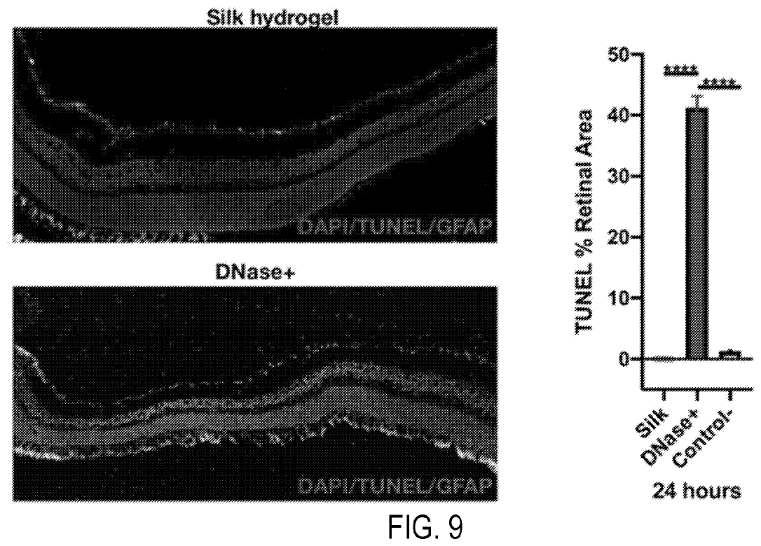

### DETAILED DESCRIPTION

In some embodiments, the present disclosure provides crosslinked silk fibroin-based compositions, methods of making, and using the same. The crosslinked silk fibroin-based compositions are useful in a number of applications, including soft tissue supplementation, soft tissue augmentation, and polymeric networks that can be utilized as scaffolds for biomedical applications. Example applications include, but are not limited to, vitreous humor substitutes, mucous replacements, joint lubricity compositions, vocal cord augmentation, and biofilm related applications.

Crosslinked Silk Fibroin-Based Compositions:

In some embodiments, the crosslinked silk fibroin-based compositions comprise silk fibroin and a glycosaminoglycan crosslinked to at least a portion of the silk fibroin.

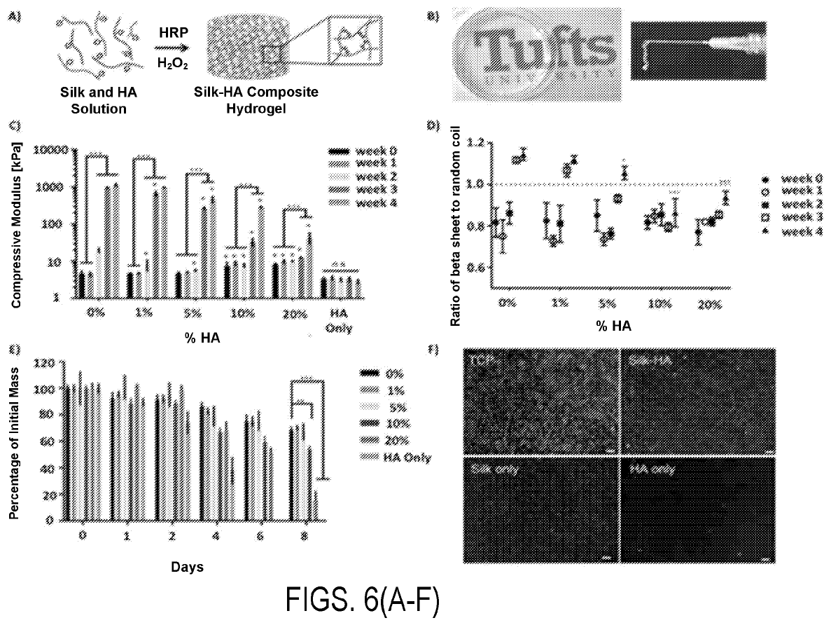

In some embodiments, the glycosaminoglycan is crosslinked to the silk fibroin through a linking agent. As used herein, the term “linking agent” may refer to an organic moiety that covalently bonds the glycosaminoglycan to the silk fibroin. As shown in FIG. 6A, in some embodiments, the linking agent is a phenol group (e.g., tyramine) on the glycosaminoglycan that forms a covalently bonds to an amino acid (e.g., tyrosine) of the silk fibroin. As will be further detailed, the covalent bond between the tyrosine and tyramine may be induced via horseradish peroxidase enzyme and hydrogen peroxide, which crosslinks the silk fibroin and glycosaminoglycan, and further induces gelation.

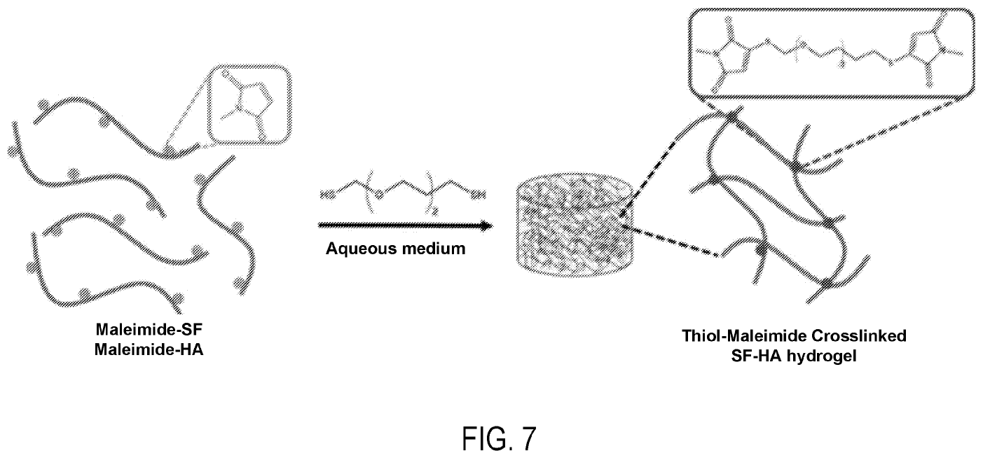

As shown in FIG. 7, in some embodiments, the linking agent includes maleimide groups that have been coupled on the surface of the glycosaminoglycan and the silk fibroin (e.g., via bioconjugation by EDC coupling). The linking agent may further include a dithiol crosslinker that bridges the maleimide groups to form a thiol-maleimide linking agent. Other click chemistry reactions may be performed between the silk fibroin and the glycosaminoglycan to promote crosslinking and gelation including, but not limited to, furan-maleimide diels-alder click chemistry and copper-free click chemistry.

In some embodiments, the crosslinked silk fibroin hydrogels include a carrier or biological fluid. In some embodiments, the carrier or biological fluid includes a solvent and/or dispersing medium. Suitable carriers and/or biological fluids include, but are not limited to, water, cell culture medium, buffers (e.g., phosphate buffered saline), a buffered solution (e.g. PBS), polyol (for example, glycerol, propylene glycol, liquid polyethylene glycol, and the like), Dulbecco's Modified Eagle Medium, HEPES, Hank's balanced medium, Roswell Park Memorial Institute (RPMI) medium, fetal bovine serum, or suitable combinations and/or mixtures thereof.

In some embodiments, the silk fibroin-based composition is a hydrogel. In some embodiments, the crosslinked silk fibroin hydrogels are designed to have properties particularly suited for soft tissue supplementation and augmentation. Namely, the crosslinked silk fibroin hydrogels may be designed for vitreous humor substitution. Hydrogels for vitreous humor substitutes should exhibit gelation rates suitable for in situ gelation (from 3 to 20 minutes) to allow for injection through small gauge needles (e.g., from 32 G to 18 G), exhibit appropriate mechanical properties (2 to >100 Pa shear storage modulus), have extended degradation rates, be biocompatible, optically clear (75-95% light transmittance, 400-700 nm), have controllable swelling, and exhibit stability over at least 6 months (time at which the clinical standard, silicone oil, should be removed to avoid toxicity).

In some embodiments, the properties of the crosslinked silk fibroin hydrogels may be modulated to achieve one or more of these properties by controlling the concentration of silk fibroin, the concentration of glycosaminoglycan, the crosslinking density between the silk fibroin and the glycosaminoglycan, and the molecular weight of both the silk fibroin and the glycosaminoglycan in the hydrogel composition. In some embodiments, the crosslinking density between the silk fibroin and the glycosaminoglycan can be modulated by adjusting the concentration of a crosslinking agent during formation of the hydrogel.

In some embodiments, the crosslinked silk fibroin hydrogels comprise a total polymer content from 0.1% weight by volume percentage (w/v) to 10% w/v, based on the total volume of the crosslinked silk fibroin hydrogel. In some embodiments, the total polymer content ranges from 0.1% w/v to 1% w/v, or 0.1% w/v to 1.5% w/v, or 0.1% to 2% w/v.

In some embodiments, the total polymer content is the sum weight by volume percentage of silk fibroin and glycosaminoglycan in the hydrogel. In some embodiments, the crosslinked silk fibroin hydrogels comprise a total polymer content of at least 0.1% w/v, or at least 0.2% w/v, or at least 0.3% w/v, or at least 0.4% w/v, or at least 0.5% w/v, or at least 0.6% w/v, or at least 0.7% w/v, or at least 0.8% w/v, or at least 0.9% w/v, or at least 1% w/v, or at least 1.1% w/v, or at least 1.2% w/v, or at least 1.3% w/v, or at least 1.4% w/v, or at least 1.5% w/v, or at least 1.6% w/v, or at least 1.7% w/v, or at least 1.8% w/v, or at least 1.9% w/v, or at least 2.0% w/v, or at least 2.5% w/v, or at least 3% w/v, or at least 3.5% w/v, or at least 4% w/v, or at least 4.5% w/v, or at least 5% w/v. In some embodiments, the crosslinked silk fibroin hydrogels comprise a total polymer content of less than 5.5% w/v, or less than 6% w/v, or less than 6.5% w/v, or less than 7% w/v, or less than 7.5% w/v, or less than 8% w/v, or less than 8.5% w/v, or less than 9% w/v, or less than 9.5% w/v, or less than 10% w/v.

In some embodiments, the crosslinked silk fibroin hydrogels comprise silk fibroin at a concentration of 0.01% w/v to 9.9% w/v, based on the total volume of the crosslinked silk fibroin hydrogel. In some embodiments, the silk fibroin concentration ranges from 0.05% w/v to 1% w/v, or 0.05% w/v to 1.5% w/v, or 0.05% to 2% w/v.

In some embodiments, the crosslinked silk fibroin hydrogels comprise silk fibroin at a concentration of at least 0.01% w/v, or at least 0.05% w/v, 0.1% w/v, or at least 0.2% w/v, or at least 0.3% w/v, or at least 0.4% w/v, or at least 0.5% w/v, or at least 0.6% w/v, or at least 0.7% w/v, or at least 0.8% w/v, or at least 0.9% w/v, or at least 1% w/v, or at least 1.1% w/v, or at least 1.2% w/v, or at least 1.3% w/v, or at least 1.4% w/v, or at least 1.5% w/v, or at least 1.6% w/v, or at least 1.7% w/v, or at least 1.8% w/v, or at least 1.9% w/v, or at least 2.0% w/v, or at least 2.5% w/v, or at least 3% w/v, or at least 3.5% w/v, or at least 4% w/v, or at least 4.5% w/v, or at least 5% w/v. In some embodiments, the crosslinked silk fibroin hydrogels comprises silk fibroin at a concentration of less than 5.5% w/v, or less than 6% w/v, or less than 6.5% w/v, or less than 7% w/v, or less than 7.5% w/v, or less than 8% w/v, or less than 8.5% w/v, or less than 9% w/v, or less than 9.5% w/v, or less than 9.9% w/v.

In some embodiments, the crosslinked silk fibroin hydrogels comprise a glycosaminoglycan at a concentration of 0.01% w/v to 9.9% w/v, based on the total volume of the crosslinked silk fibroin hydrogel. In some embodiments, the crosslinked silk fibroin hydrogels comprise the glycosaminoglycan at a concentration of 0.05% w/v to 9.9% w/v, based on the total volume of the crosslinked silk fibroin hydrogel. In some embodiments, the silk fibroin concentration ranges from 0.05% w/v to 1% w/v, or 0.05% w/v to 1.5% w/v, or 0.05% to 2% w/v.

In some embodiments, the crosslinked silk fibroin hydrogels comprise the glycosaminoglycan at a concentration of at least 0.01% w/v, or at least 0.05% w/v, or at least 0.1% w/v, or at least 0.2% w/v, or at least 0.3% w/v, or at least 0.4% w/v, or at least 0.5% w/v, or at least 0.6% w/v, or at least 0.7% w/v, or at least 0.8% w/v, or at least 0.9% w/v, or at least 1% w/v, or at least 1.1% w/v, or at least 1.2% w/v, or at least 1.3% w/v, or at least 1.4% w/v, or at least 1.5% w/v, or at least 1.6% w/v, or at least 1.7% w/v, or at least 1.8% w/v, or at least 1.9% w/v, or at least 2.0% w/v, or at least 2.5% w/v, or at least 3% w/v, or at least 3.5% w/v, or at least 4% w/v, or at least 4.5% w/v, or at least 5% w/v. In some embodiments, the crosslinked silk fibroin hydrogels comprises silk fibroin at a concentration of less than 5.5% w/v, or less than 6% w/v, or less than 6.5% w/v, or less than 7% w/v, or less than 7.5% w/v, or less than 8% w/v, or less than 8.5% w/v, or less than 9% w/v, or less than 9.5% w/v, or less than 9.9% w/v.

In some embodiments, the silk fibroin and glycosaminoglycan are present in the crosslinked silk fibroin hydrogel at a ratio from 1:99 to 99:1 (silk fibroin:glycosaminoglycan). In some embodiments, the ratio is 1:99 (silk fibroin:glycosaminoglycan), or 5:95, or 10:90, or 15:85, or 20:80, or 25:75, or 30:70, or 35:65, or 40:60, or 45:55, or 50:50, or, 55:45, or 60:40, or 65:35, or 70:30, or 75:25, or 80:20, or 85:15, or 90:10, or 95:5, or 99:1.

In some embodiments, the crosslinked silk fibroin hydrogels have a shear storage modulus (G′) from 6 to 4000 Pa. In some embodiments, the storage modulus is modulated based on the total polymer content, the ratio of silk fibroin to the glycosaminoglycan, and the crosslinking density. In some embodiments, the shear storage modulus may be at least 6 Pa, at least 10 Pa, at least 20 Pa, at least 30 Pa, at least 40 Pa, at least 50 Pa, at least 60 Pa, at least 70 Pa, at least 80 Pa, at least 90 Pa, at least 100 Pa, at least 110 Pa, at least 150 Pa, at least 200 Pa, at least 300 Pa, at least 400 Pa, at least 500 Pa, at least 600 Pa, at least 700 Pa, at least 800 Pa, at least 900 Pa, or at least 1000 Pa. In some embodiments, the shear storage modulus is less than 1500 Pa, is less than 2000 Pa, is less than 2500 Pa, is less than 3000 Pa, is less than 3500 Pa, or is less than 4000 Pa.

In some embodiments, the injectable composition is adapted to provide a hydrogel stability by maintaining at least 75%, or at least 80%, or at least 85%, or at least 90%, or at least 95% of the storage modulus of the hydrogel between 6 to 4000 Pa after for at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least one year. In some embodiments, the storage modulus remains between 6 to 4000 Pa when the crosslinked silk fibroin hydrogel is implanted under physiological conditions for the aforementioned durations. In some embodiments, the storage modulus remains between 6 to 4000 Pa when the crosslinked silk fibroin hydrogel is placed in an aqueous or saline solution at 37° C. for the aforementioned durations.

As used herein, the term “physiological conditions” has its art-understood meaning referencing conditions under which cells or organisms live and/or reproduce. In some embodiments, the term refers to conditions of the external or internal mileu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

In some embodiments, the crosslinked silk fibroin hydrogels have an in situ gelation time that ranges from 3 to 20 minutes. Gelation times within this specified range are particularly advantageous for vitreous humor substitutes as it allows for sufficient mixing time and in situ formation of the gels on injection. Gelation times within this range also allow for injection through small gauge needles (e.g., 32G needle, 30G needle, 28G needle, 26G needle, 24 G needle, 22G needle, 20G needle, or a 18G needle). In some embodiments, the gelation is tuned through the silk fibroin and glycosaminoglycan ratio and crosslinking density.

In some embodiments the in situ gelation time is at least 3 minutes, is at least 4 minutes, is at least 5 minutes, is at least 6 minutes, is at least 7 minutes, is at least 8 minutes, is at least 9 minutes, or is at least 10 minutes. In some embodiments, the in situ gelation time is less than 11 minutes, is less than 12 minutes, is less than 13 minutes, is less than 14 minutes, is less than 15 minutes, is less than 16 minutes, is less than 17 minutes, is less than 18 minutes, is less than 19 minutes, or is less than 20 minutes.

In some embodiments, the crosslinked silk fibroin hydrogels are optically clear. In some embodiments, the crosslinked silk fibroin hydrogels have a visible light transmittance from 70% to 95%. In some embodiments, the transparency of the hydrogels may be determined by the percentage of light transmitted for wavelengths within the visible range (e.g., 400 nm to 700 nm), using a SpectraMax M2 multi-mode microplate reader. Reduced transmittance at lower wavelengths is advantageous for vitreous humor substitutes as it helps to prevent damage to retinal epithelium.

In some embodiments, the crosslinked silk fibroin hydrogels are adapted to provide a hydrogel stability by maintaining at least 75%, or at least 80%, or at least 85%, or at least 90%, or at least 95% of the optical transmittance of the hydrogel after a duration. In some embodiments, the visible light transmittance remains between 70% to 95% after at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least one year. In some embodiments, the visible light transmittance remains between 70% to 95% when the crosslinked silk fibroin hydrogel is implanted under physiological conditions for the aforementioned durations. In some embodiments, the visible light transmittance remains between 70% to 95% when the crosslinked silk fibroin hydrogel is placed in an aqueous or saline solution at 37° C. for the aforementioned durations.

In some embodiments, the crosslinked silk fibroin hydrogels have tunable swelling properties. The degree of swelling may be modulated through the ratio of silk fibroin to glycosaminoglycan and the crosslinking density (e.g., concentration of the crosslinking agent). In some embodiments, the crosslinked silk fibroin hydrogels expand from 5% to 400% of the original volume when placed in an aqueous solution or a saline solution at 37° C.

In some embodiments, the crosslinked silk fibroin hydrogels volume expands at least 5%, at least 50%, at least 100%, at least 150%, or at least 200% of their original volume when placed in an aqueous or buffered solution at 37° C. In some embodiments, the crosslinked silk fibroin hydrogels expand up to 250%, or up to 300%, or up to 350%, or up to 400% of their original volume. In some embodiments, the swelling occurs over a duration of at least 12 hours, or one day, or at least 2 days, or at least 3 days, or at least 4 days, or at least 5 days, or at least 6 days, or at least one week, or at least two weeks, or at least three weeks, or at least four weeks, or at least one month, or at least 6 months, or at least one year. In some embodiments, the crosslinked silk fibroin hydrogels example from 5% to 400% of their original volume under physiological conditions during the course of the aforementioned durations.

In some embodiments, provided compositions may exhibit improved swelling properties. Specifically, in some embodiments, provided compositions may exhibit a mass fraction of at most 1.00 after soaking in an aqueous solution for 12 hours. In some embodiments, provided compositions may have mass fractions of between 0.4-0.99 after soaking in an aqueous solution for 12 hours. In some embodiments, provided compositions may have mass fractions of greater than 1.00 (e.g., greater than 1.1, 1.2. 1.3, 1.4, 1.5, 2.0, 2.5, 5.0, etc) after soaking in an aqueous solution for 12 hours. In some embodiments, provided compositions may exhibit a mass fraction of between 0.4 and 5.0 after soaking in an aqueous solution for 12 hours.

As used herein, the term “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein as reference in its entirety.

Thus, in some embodiments, a silk solution is used to fabricate compositions of the present invention contain fibroin proteins, essentially free of sericins. In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins.

In certain embodiments, silk solutions used to fabricate various compositions of the present disclosure comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

In accordance with various embodiments, silk fibroin may be modified prior to use in provided methods and compositions. For example, in some embodiments, silk fibroin may be modified to include more tyrosine groups than native silk fibroin (e.g., silk fibroin from a silkworm or a spider), or to include fewer tyrosine groups than native silk fibroin. By way of specific example, in some embodiments, silk fibroin may be modified to include at least one non-native tyrosine. In some embodiments, silk fibroin may be modified to remove or alter at least one tyrosine group from native silk fibroin such that it is no longer able to crosslink with a phenol-containing polymer. In some embodiments, addition of tyrosine groups to silk fibroin may occur via carbodiimide chemistry. In some embodiments, the silk fibroin may have an enriched tyrosine content relative to the native silk. In some embodiments, the silk fibroin in the crosslinked silk fibroin hydrogel may have an enriched tyrosine content. In some embodiments, the crosslinked silk fibroin hydrogel has an enriched tyrosine content of at least 1%, or at least 2%, or at least 3%, or at least 4%, or at least 5%, or at least 6%, or at least 7%, or at least 8%, or at least 9%, or at least 10% relative to native silk.

Any known technique for quantifying the amount of tyrosine groups on silk fibroin may be used. For example, in some embodiments, quantification of tyrosine groups may be performing using one or more of spectrophotometric analysis (e.g., via UV absorbance), liquid chromatography-mass spectrometry (LC-MS), and high performance liquid chromatography (HPLC). This can be performed before and after modified to determine the enriched tyrosine content.

In some embodiments, silk fibroin fragments may be of any application-appropriate size. For example, in some embodiments, silk fibroin fragments may have a molecular weight of 200 kDa or less (e.g., less than 190 kDa, less than 180 kDa, less than 170 kDa, less than 160 kDa, less than 150 kDa, less than 140 kDa, less than 130 kDa, less than 125 kDa, less than 100 kDa, less than 75 kDa, or less than 50 kDa). Without wishing to be held to a particular theory, it is contemplated that the size of silk fibroin fragments may impact gelation time and rate of crosslinking. By way of specific example, in some embodiments, use of silk fragments of a relatively low molecular weight (e.g., less than 200 kDa) may result in relatively more rapid crosslinking due, at least in part, to the greater mobility of the available chains for reacting in a crosslinking step. Adjusting the molecular weight of silk fibroin may be used to attain the desired properties of the crosslinked silk fibroin hydrogel described herein.

As used herein, the term “glycosaminoglycan” may refer to polysaccharides consisting of repeating disaccharide units. In some embodiments, the glycosaminoglycan is hyaluronic acid. The glycosaminoglycan may be surface modified to have an enhanced phenol group (e.g., tyramine) content. In some embodiments, the glycosaminoglycan is surface modified to have an enriched phenol group content from 1% to 10% greater than the glycosaminoglycan without the modification. In some embodiments, the glycosaminoglycan has an enriched phenol group content of at least 1%, or at least 2%, or at least 3%, or at least 4%, or at least 5%. In some embodiments, the glycosaminoglycan has an enriched phenol group content of up to 6%, or up to 7%, or up to 8%, or up to 9%, or up to 10%.

In some embodiments, the glycosaminoglycan has a molecular weight from 500 kDa to 5000 kDa. In some embodiments, the glycosaminoglycan has a molecular weight of at least 500 kDa, of at least 600 kDa, of at least 700 kDa, of at least 800 kDa, of at least 900 kDa, of at least 1000 kDa, of at least 1100 kDa, of at least 1200 kDa, of at least 1300 kDa, of at least 1400 kDa, of at least 1500 kDa. In some embodiments, the molecular weight is less than 1600 kDa, or less than 1700 kDa, or less than 1800 kDa, or less than 1900 kDa, or less than 2000 kDa, or less than 3000 kDa, or less than 4000 kDa. In some aspects, using higher molecular weight hyaluronic acid results in hydrogels having an improved noninflammatory response for vitreous humor substitutes (e.g. greater than 1000 kDa).

In some embodiments, provided compositions (e.g., hydrogels) can comprise one or more (e.g., one, two, three, four, five or more) active agents and/or functional moieties (together, “additives”). Without wishing to be bound by a theory, an additive can provide or enhance one or more desirable properties, e.g., strength, flexibility, ease of processing and handling, biocompatibility, bioresorability, surface morphology, release rates and/or kinetics of one or more active agents present in the composition, and the like. In some embodiments, one or more such additives can be covalently or non-covalently linked with a composition (e.g., with a polymer such as silk fibroin that makes up the hydrogel) and can be integrated homogenously or heterogeneously (e.g., in a gradient or in discrete portions of a provided composition) within the silk composition.

In some embodiments, provided compositions (e.g., hydrogels) comprise additives at a total amount from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the additive comprises a sensing agent or reporter molecule. The sensing agents/sensing dyes are environmentally sensitive and produce a measurable response to one or more environmental factors. In some aspects, the environmentally-sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the ink composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensing agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-([-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some embodiments, provided compositions (e.g., hydrogels) comprise additives, for example, therapeutic, preventative, and/or diagnostic agents.

In some embodiments, an additive is or comprises one or more therapeutic agents. In general, a therapeutic agent is or comprises a small molecule and/or organic compound with pharmaceutical activity (e.g., activity that has been demonstrated with statistical significance in one or more relevant pre-clinical models or clinical settings). In some embodiments, a therapeutic agent is a clinically-used drug. In some embodiments, a therapeutic agent is or comprises an cells, proteins, peptides, nucleic acid analogues, nucleotides, oligonucleotides, nucleic acids (DNA, RNA, siRNA), peptide nucleic acids, aptamers, antibodies or fragments or portions thereof, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, hormones, hormone antagonists, growth factors or recombinant growth factors and fragments and variants thereof, cytokines, enzymes, antibiotics or antimicrobial compounds, antifungals, antivirals, toxins, prodrugs, chemotherapeutic agents, small molecules, drugs (e.g., drugs, dyes, amino acids, vitamins, antioxidants), pharmacologic agents, and combinations thereof.

In some embodiments, provided compositions comprise an anesthetic. Suitable anesthetics include, but are not limited to, ester-based (e.g., procaine, amethocaine, benzocaine, tetracaine) or amide-based anesthetics (lidocaine, prilocaine, bupivicaine, levobupivacaine, ropivacaine, mepivacaine, dibucaine, and etidocaine), or combinations thereof.

In some embodiments, provided compositions (e.g., hydrogels) comprise additives, for example, cells. In some embodiments, methods of using provided compositions may comprise adhering cells to a surface of a covalently crosslinked hydrogel. In some embodiments, methods of using provided compositions may comprise encapsulating cells within a matrix a covalently crosslinked hydrogel. In some embodiments, methods of using provided compositions may comprise encapsulating cells for introducing cells to a native tissue. Cells suitable for use herein include, but are not limited to, progenitor cells or stem cells, smooth muscle cells, skeletal muscle cells, cardiac muscle cells, glial cells (e.g., astrocytes), neurons, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

In some embodiments, provided compositions (e.g., hydrogels) comprise additives, for example, organisms, such as, a bacterium, fungus, plant or animal, or a virus. In some embodiments, an active agent may include or be selected from neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

In some embodiments, provided compositions (e.g., hydrogels) comprise additives, for example, antibiotics. Antibiotics suitable for incorporation in various embodiments include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, β-lactam antibiotics, rifamycins, novobiocin, fusidate sodium, capreomycin, colistimethate, gramicidin, doxycycline, erythromycin, nalidixic acid, and vancomycin. For example, β-lactam antibiotics can be aziocillin, aztreonam, carbenicillin, cefoperazone, ceftriaxone, cephaloridine, cephalothin, moxalactam, piperacillin, ticarcillin and combination thereof.

In some embodiments, provided compositions (e.g., hydrogels) comprise additives, for example, anti-inflammatories. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

In some embodiments, provided compositions (e.g., hydrogels) comprise additives, for example, antibodies. Suitable antibodies for incorporation in hydrogels include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, lab etuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

In some embodiments, provided compositions (e.g., hydrogels) comprise additives, for example, polypeptides (e.g., proteins), including but are not limited to: one or more antigens, cytokines, hormones, chemokines, enzymes, and any combination thereof as an agent and/or functional group. Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

In some embodiments, provided compositions (e.g., hydrogels) comprise additives, for example, particularly useful for wound healing. In some embodiments, agents useful for wound healing include stimulators, enhancers or positive mediators of the wound healing cascade (e.g., wound healing growth factors) which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

In some embodiments, provided compositions (e.g., hydrogels) comprise additives, for example, an optically or electrically active agent, including but not limited to, chromophores; light emitting organic compounds such as luciferin, carotenes; light emitting inorganic compounds, such as chemical dyes; light harvesting compounds such as chlorophyll, bacteriorhodopsin, protorhodopsin, and porphyrins; light capturing complexes such as phycobiliproteins; and related electronically active compounds; and combinations thereof.

In some embodiments, provided compositions (e.g., hydrogels) comprise additives, for example, nucleic acid agents. In some embodiments, a composition may release nucleic acid agents. In some embodiments, a nucleic acid agent is or comprises a therapeutic agent. In some embodiments, a nucleic acid agent is or comprises a diagnostic agent. In some embodiments, a nucleic acid agent is or comprises a prophylactic agent.

In some embodiments, provided compositions (e.g., hydrogels) comprise additives, for example, one or more growth factors. In some embodiments, a provided composition may release one or more growth factors. In some embodiments, a provided composition may release multiple growth factors. In some embodiments growth factors known in the art include, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof

Method of Forming the Crosslinked Silk Fibroin-Based Compositions

In some aspects, provided methods may comprise contacting a silk fibroin solution and a glycosaminoglycan solution with a crosslinking agent to induce crosslinking between at least a portion of the silk fibroin and the glycosaminoglycan. In some embodiments, the crosslinking agent is utilized at a concentration of 0.01% weight by weight (w/w) to 5% (w/w), based on the total weight of the composition. In some embodiments, the crosslinking agent is utilized at a concentration from 0.1% (w/w) to 0.5% (w/w), from 0.1% (w/w) to 1% (w/w), or 0.1% (w/w) to 3% (w/w).

In some embodiments, the crosslinking agent comprises an enzyme and an oxidizing agent. Suitable enzymes include, but are not limited to, horseradish peroxidase, and suitable oxidizing agents include, but are not limited to, hydrogen peroxide. Horseradish peroxidase and hydrogen peroxide induce enzymatic crosslinking between amino acid side chains of silk fibroin (e.g., tyrosine) and phenol groups in the glycosaminoglycan. The gel initiation and gelation rate and/or kinetic properties of the process can be tunable or controlled, for example, depending on concentrations of silk, enzyme (e.g., HRP), and/or substrate for the enzyme (e.g., H₂O₂).

In some embodiments, the enzyme is utilized at a concentration of 0.01 U/mL to 10 U/mL. In some embodiments, the enzyme is utilized at a concentration of at least 0.01 U/mL, or at least 0.1 U/mL, or at least 0.5 U/mL, or at least 1 U/mL, or at least 1.5 U/mL, or at least 2 U/ml, or at least 2.5 U/mL, or at least 3 U/mL, or at least 3.5 U/mL, or at least 4 U/mL, or at least 4.5 U/mL, or at least 5 U/mL, or at least 5.5 U/mL, or at least 6 U/mL. In some embodiments, the enzyme is utilized at a concentration of less than 6.5 U/mL, or less than 7 U/mL, or less than 7.5 U/mL, or less than 8 U/mL, or less than 8.5 U/mL, or less than 9 U/mL, or less than 9.5 U/mL, or less than 10 U/mL.

In some embodiments, the oxidizing agent is utilized at a concentration from 0.001% volume by volume (v/v) to 3% (v/v). In some embodiments the oxidizing agent is utilized at a concentration of at least 0.001% (v/v), or at least 0.01% (v/v), or at least 0.1% (v/v), or at least 0.5% (v/v), or at least 1% (v/v). In some embodiments, the oxidizing agent is utilized at a concentration of less than 1% (v/v), or less than 2% (v/v), or less than 3% (v/v).

In some embodiments, the crosslinking agent comprises a reactive organic moiety that induces crosslinking between the glycosaminoglycan and the silk fibroin. In one embodiment, the silk fibroin and the glycosaminoglycan can be surface modified to include a surface modification that facilitates crosslinking. In some embodiments, the surface modification includes one or more chemical moiety selected from a sulfonic acid group, a carboxylic acid group, an amine group, a ketone group, an alkyl group (e.g., branched or linear C₁-C₂₀), an alkoxy group, a thiol group, a disulfide group, a nitro group, an aromatic group, an ester group, an amide group, and a hydroxyl group. The surface modification may be coupled to the surface of the glycosaminoglycan using any suitable methods, for example, bioconjugation via EDC coupling or diazonium salts.

In some embodiments, a reactive organic moiety is reacted with the surface modified to induce gelation and crosslinking between the glycosaminoglycan and the silk fibroin. In some embodiments, the reactive organic moiety comprises one or more chemical moiety selected from a sulfonic acid group, a carboxylic acid group, an amine group, a ketone group, an alkyl group (e.g., branched or linear C₁-C₂₀), an alkoxy group, a thiol group, a disulfide group, a nitro group, an aromatic group, an ester group, an amide group, and a hydroxyl group. In one non-limiting example, the reactive moiety may include any organic compound that reacts with the maleimide groups to form a covalent bond between the silk fibroin and the glycosamingoglycan. In some embodiments, the reactive organic moiety is a dithiol organic compound.

Silk materials explicitly exemplified herein were typically prepared from material spun by silkworm, Bombyx mori. Typically, cocoons are boiled in an aqueous solution of 0.02 M Na2 CO3, then rinsed thoroughly with water to extract the glue-like sericin proteins (this is also referred to as “degumming” silk). Extracted silk is then dissolved in a solvent, for example, LiBr (such as 9.3 M) solution at room temperature. A resulting silk fibroin solution can then be further processed for a variety of applications as described elsewhere herein.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, hydrogels of the present invention produced from silk fibroin fragments can be formed by degumming silk cocoons in an aqueous solution at temperatures of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C.

In some embodiments, the silk fibroin and the glycosaminoglycan may be solubilized prior to gelation. In some embodiments, a carrier can be a solvent or dispersing medium. In some embodiments, a solvent and/or dispersing medium, for example, is water, cell culture medium, buffers (e.g., phosphate buffered saline), a buffered solution (e.g. PBS), polyol (for example, glycerol, propylene glycol, liquid polyethylene glycol, and the like), Dulbecco's Modified Eagle Medium, HEPES, Hank's balanced medium, Roswell Park Memorial Institute (RPMI) medium, fetal bovine serum, or suitable combinations and/or mixtures thereof.

In some embodiments, the properties of provided compositions may be modulated by controlling a concentration of silk fibroin and the glycosaminoglycan. In some embodiments, the method includes controlling the total polymer content in solution to be from 0.1% weight by volume percentage (w/v) to 10% w/v, based on the total volume of the crosslinked silk fibroin hydrogel. In some embodiments, the total polymer content ranges from 0.1% w/v to 1% w/v, or 0.1% w/v to 1.5% w/v, or 0.1% to 2% w/v.

In some embodiments, the solutions may include silk fibroin at a concentration of 0.1% w/v to 9.9% w/v. In some embodiments, the solutions may include the glycosaminoglycan at a concentration of 0.1% w/v to 9.9% w/v. The glycosaminoglycan and the silk fibroin may be reacted with the crosslinking agents at these concentrations to induce gelation and crosslinking.

### EXAMPLES

The following examples set forth, in detail, ways in which the compositions described herein may be synthesized, and will enable one of skill in the art to more readily understand the principles thereof. The following examples are presented by way of illustration and are not meant to be limiting in any way.

### Example 1

An Exemplary Method for the Crosslinked Silk Fibroin Hydrogels

Preparation of Aqueous Silk Fibroin:

Silk fibroin was isolated from Bombyx mori silkworm cocoons. Cut cocoons were degummed in boiling sodium carbonate (Sigma-Aldrich, St. Louis, Mo.) solution for 60 minutes. The resulting fibers were washed in deionized (DI) water and then dissolved in 9.3M lithium bromide (Sigma-Aldrich, St. Louis, Mo.) for 4 hours. The resulting silk fibroin solution was dialyzed against water for 3 days to remove residual lithium bromide and diluted to 4% w/v using DI or ultrapure water.

Hydrogel Formation:

Lyophilized hyaluronic acid (HA) with a 5% tyramine substitution (Corgel® powder, Lifecore Biomedical, Chaska, Minn.) was dissolved in hank's balanced salt solution (HBSS; without magnesium and calcium; ThermoFisher Scientific, Waltham, Mass.) at a concentration of 10-11 mg/mL. Silk and HA were combined with varying silk/HA (S/H) ratios (50S/50H, 25S/75S, 10S/90H) at a total concentration of 1% w/v of polymer using HBSS as dilutant. Higher ratios of silk were not utilized due to lack of gelation. Silk and HA were covalently crosslinked through enzymatic means.

Table 1 specifies the crosslinking conditions for each sample. HRP (Type VI, Sigma-Aldrich, St. Louis, Mo.) concentration was adjusted to obtain gelation times between 5-15 minutes (Table 2). Three H₂O₂ (Sigma-Aldrich, St. Louis, Mo.) concentrations (low, medium, and high) were chosen for each of the ratios to alter crosslinking density resulting in an average shear storage moduli of less than 30 Pa, 30-110 Pa, and above 110 Pa. HA control hydrogels were formed using at a ratio of 0S/100H and used the same crosslinking conditions as the 10S/90H ratio. Solutions were either kept sterile or filter sterilized prior to gelation. Components were mixed on ice and allowed to gel for 1 hour at 37° C. prior to submerging in HBSS (with magnesium and calcium) unless otherwise mentioned.

TABLE 1
Crosslinking conditions for samples. All hydrogels contain 1%
w/v polymer in HBSS with varying silk/hyaluronic acid (S/H) ratios.

| | | H₂O₂ | HRP |
|---|---|---|---|
| Sample | | (x10⁻² % v/v) | (U/mL) |
| 50S/50H | Low | 0.05 | 1 |
| | Med | 0.1 | |
| | High | 0.3 | |
| 25S/75H | Low | 0.01 | 0.1 |
| | Med | 0.05 | |
| | High | 0.1 | |
| 10S/90H | Low | 0.01 | 0.05 |
| 0S/100H | Med | 0.02 | |
| | High | 0.05 | |

TABLE 2
Hydrogel gelation was optimized by altering HRP concentration for
polymer ratios to facilitate gelation time between 10-15 minutes at 37° C.
Gelation time was determined when the solution was no longer pipettable.
(NR = not recorded).

| | | Gelation time | |
|---|---|---|---|
| | HRP Concentration | (minutes) | |
| Ratio | (U/mL) | On ice | 37° C. |
| 50S/50H | 1 | 17-18 | 12-13 |
| 25S/75H | 0.1 | 30 | 10 |
| 10S/90H | 0.01 | >40 | >40 |
| | 0.05 | NR | 11-12 |
| | 0.08 | NR | 5-6 |

Rheological Measurements:

Hydrogels were allowed to gel in 60 mm petri dishes for rheological analysis. Rheological properties were assessed on hydrogels after incubation in HBSS at 37° C. for 1 and 30 days using a TA Instruments ARES-LS2 rheometer (TA Instruments, New Castle, Del.) fitted with a 50 mm stainless steel upper plate and peltier bottom plate lined with 600 grit sandpaper (McMaster-Carr, 47185A51) to prevent slippage. A preload of 0.2 N to ensure complete contact, excess hydrogel was trimmed, and silicone oil was placed around the plate to prevent evaporation during analysis. A dynamic single point test was performed at 1 Hz with 3% applied strained followed by dynamic frequency sweeps (0.1-100 rad/s at 3% strain) and strain sweeps (0.1%-500%, at 1 Hz) to ensure linear viscoelasticity.

Optical Measurements:

The refractive index of the hydrogels were assessed by placing a thin layer of hydrogel precursor solution or HBSS solution control onto the plate of a DANOPLUS 3 in 1 Scale Clinical Refractometer with ATC. After 20 minutes at room temperature to ensure hydrogel formation, the refractive index was measured as per manufacturer instructions. The transparency of the hydrogels was characterized. In brief, 100 μL of hydrogel solution was placed and allowed to gel in a clear 96-well plate and incubated in HBSS for 1 day and 30 days. The percentage of light transmitted through the hydrogel for wavelengths in the visible range (400-700 nm) was determined using a SpectraMax M2 multi-mode microplate reader (Molecular Devices, Sunnyvale, Calif.).

Swelling Measurements:

Hydrogel cylindrical plugs were prepared by placing hydrogel solution into 8 mm pluronic-F127 coated PDMS molds. After 1-2 hours incubating at 37° C., hydrogel plugs were removed from of the molds and incubated in HBSS at 37° C. for 1 month. Volumetric measurements were performed via calipers prior to incubation and after 1 day, 7 days, 15 days, and 29 days. To compare different S/H ratios at the same H₂O₂ concentrations, the volume was normalized to initial measurements.

Statistical Analysis:

Data are expressed as means±standard deviations. To determine statistical significance (p≤0.05) for rheological (n=3-4) and swelling (n=5) data, two-way ANOVA (analysis of variance) with Tukey's post hoc multiple comparisons were performed using GraphPad Prism (GraphPad Software, La Jolla, Calif.). For optical transparency (n=5), data were analyzed through two-way ANOVA with Tukey post hoc multiple comparisons and comparisons over time were determined through multiple t tests with the Holm-Sidak multiple comparison method.

Mechanical Properties:

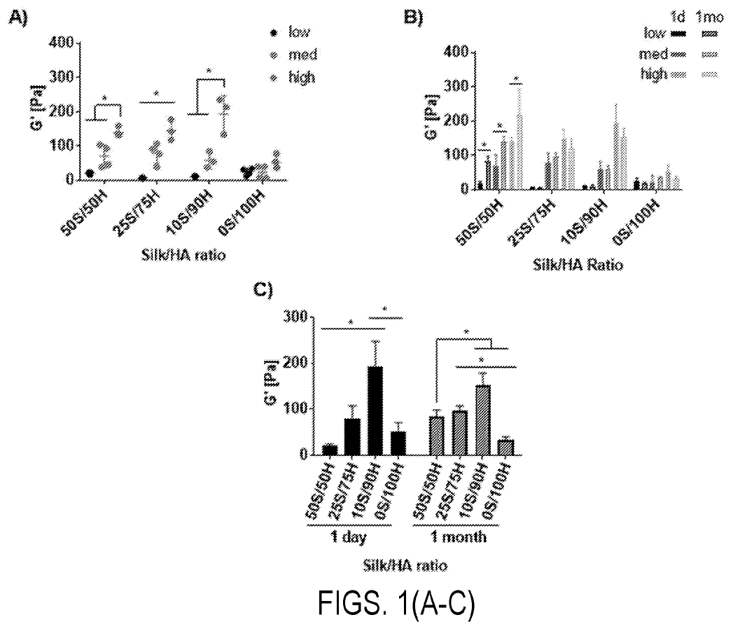

The mechanical properties of silk-HA hydrogels at varying S/H ratios and H₂O₂ concentrations were determined initially and at 1 month of incubation in physiological conditions. Concentrations of H₂O₂ were chosen for each ratio resulting in an initial shear storage modulus below 30 Pa (low), between 30-110 Pa (med), and above 110 Pa (high) (FIG. 1A). H₂O₂ concentration of the control hydrogels, consisting of HA only (0S/100H) with the sample conditions as the 10S/90S samples, did not significantly affect hydrogel modulus. After 1 month, only the 50S/50H samples for all H₂O₂ concentrations exhibited a significant increase in modulus (FIG. 1B). Specifically, there was approximately a 4×, 2×, and 1.6× increase for the low, medium, and high H₂O₂ concentrations, respectively.

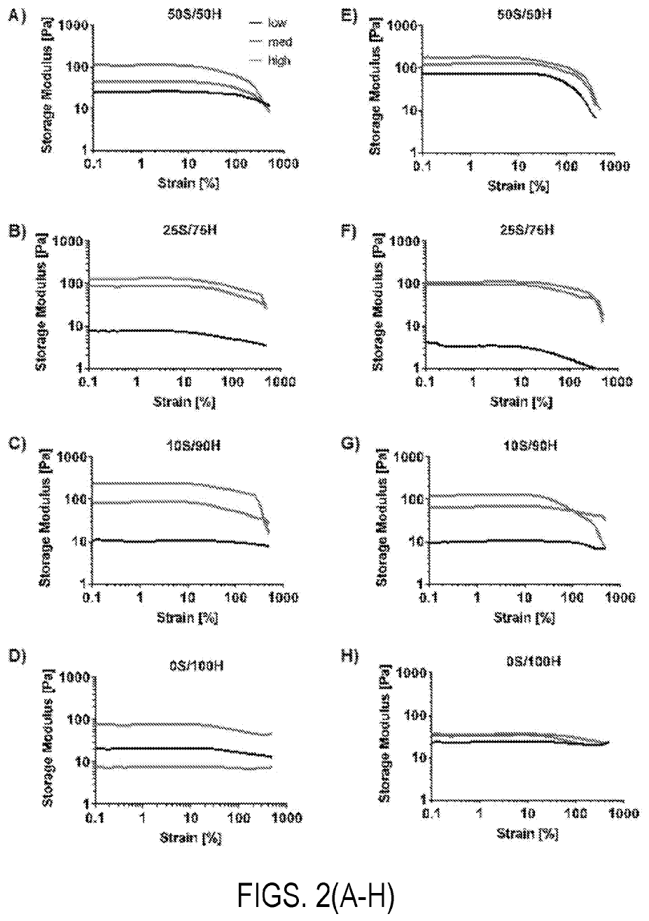

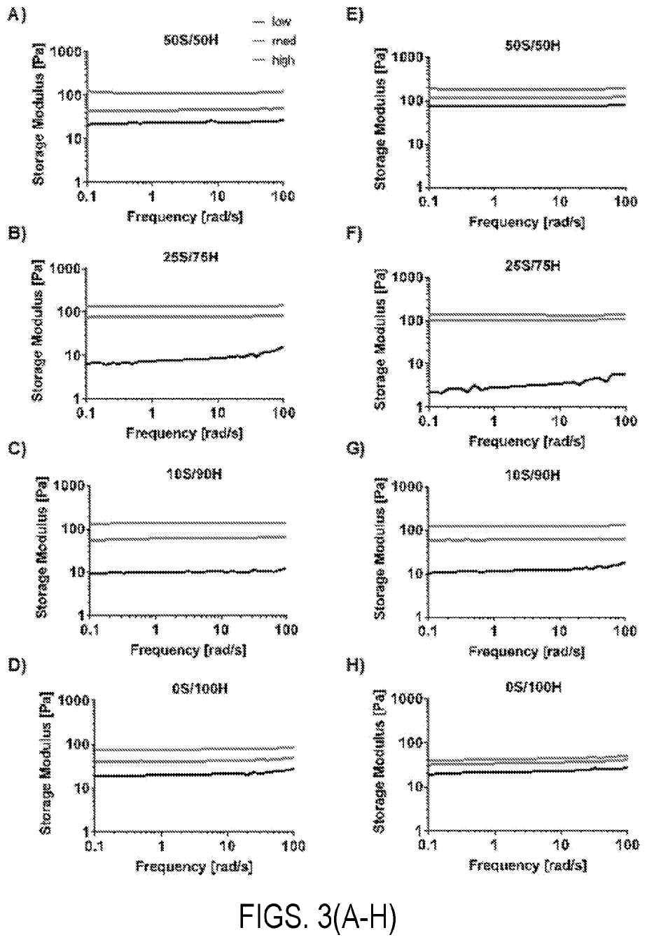

When comparing between different S/H ratios of the same S/H ratios of the same H₂O₂ concentration (Table 1), samples containing both silk and HA exhibited higher moduli with increasing HA concentration (FIG. 1C). Hydrogels with HA only controls (0S/100H) had significantly lower modulus than 10S/90H hydrogels. After 1 month, there was a similar trend with 50S/50H and 25/75H hydrogels having lower moduli than that of the 10S/90H hydrogels. In addition, at 1 month, 50S/50H hydrogels were no longer different in shear storage modulus than that of 25S/75H hydrogels. All hydrogels had linear viscoelastic regions at 3% strain (FIGS. 2A-H) and were independent of frequency except for low H₂O₂ concentration for 25S/75H, 10S/90H, and 0S/100H, which showed a slight increase in storage modulus at higher frequencies (FIGS. 3A-H).

Optical Properties:

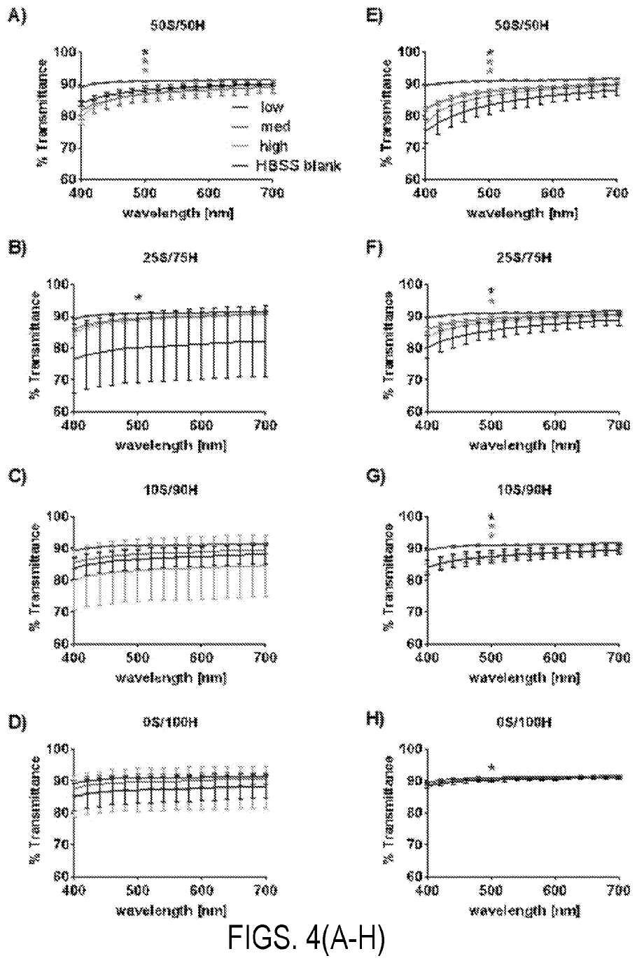

All hydrogels, regardless of S/H ratio and H₂O₂ concentration, exhibited a refractive index of 1.336 compared to HBSS that has a refractive index of 1.335. The percentage of light transmitted through the hydrogels for after 1 day and 1 month in physiological conditions are shown in (FIGS. 4A-H). Differences between the hydrogels and the HBSS control at 500 nm are shown on the graphs. Initially the percentage of light transmittance was between 76-91% depending on wavelength (FIGS. 4A-D).

The 25S/75H low hydrogels exhibited significant differences compared to the HBSS control for all wavelengths in the visible spectra (400-700 nm) (FIG. 4B). At 1 month, the optical transparency ranged between 75-91% (FIG. 4E-H) with only the 50S/50H low samples differing in transparency between day 1 and 1 month between 400-440 nm (FIGS. 4A and 4E). All hydrogels had some differences from the HBSS blank at 1 month, depending on wavelength. Samples that showed differences from the control for all wavelengths within the visible spectra (400-700 nm) at 1 month include 50S/50H low, 25S/75H low, and all of the 10S/90H H₂O₂ concentrations.

Swelling Properties:

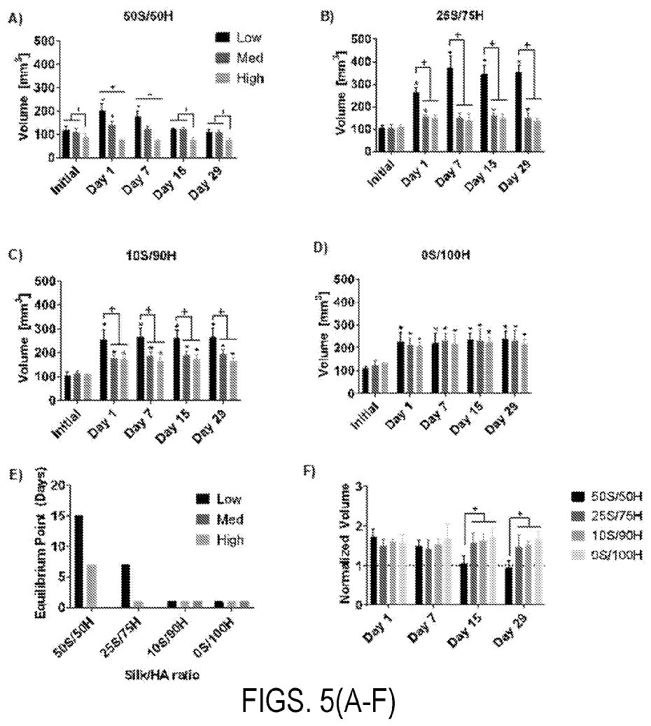

Hydrogel swelling was calculated via volumetric changes initially and up to 1 month after incubating in HBSS at 37° C. (FIGS. 5A-F). For 50S/50H ratio hydrogels, low and medium H₂O₂ concentrations resulted in increased volume after 1 day, with the volume returning to the initial values after 15 and 7 days, respectively. For the 50S/50H high H₂O₂ concentration, the hydrogel volume did not change over 1 month. Similarly, 25S/75S ratio hydrogel with high H₂O₂ concentration did not exhibit volumetric changes, whereas low and medium concentrations had sustained volumetric increase throughout 1 month with volumes equilibrating at days 1 and 7 at approximately 3.6× and 1.5× the initial volume, respectively.

For both 10S/90H and 0S/100H at all H₂O₂ concentrations, the volume increased and equilibrated at day 1. For 10S/90H samples at day 1, the samples were ˜2.5×, 1.6×, and 1.6× their initial volume for low, medium, and high H₂O₂ concentrations, respectively. For the 0S/100H samples, the volumes were 2.1×, 1.8×, and 1.6× their initial volume for low, medium, and high H₂O₂, respectively. Comparing between ratios for the same H₂O₂ concentrations, at days 15 and 29, the 50S/50H hydrogels had a significantly lower normalized volume to all other samples.

### SUMMARY

In Example 1, enzymatically crosslinked silk-HA hydrogels were characterized in the context of vitreous humor substitutes. The Example explored how altering S/H ratios and H₂O₂ concentration effected mechanical, swelling, and optical properties over time. In situ gelation from 5 minutes to 15 minutes is advantageous for polymeric vitreous substitutes as it prevents fragmentation and preserves elasticity and mechanical properties. Therefore, enzyme concentrations for each polymer ratio were chosen to allow gelation to occur within 5-15 minutes, providing adequate time for proper mixing ex vivo, and rapid gelation in vivo, to prevent migration from the delivery site. Without being bound to a particular theory, it is contemplated that altering the enzyme concentration independently controls gelation rate without affecting crosslinking density. Additionally, without being bound to a particular theory, using the HRP/H₂O₂ reaction, crosslinking density of phenol-conjugated polymers is independently tuned via altering H₂O₂ concentrations.

Ideal vitreous substitutes are biocompatible and also mimic native vitreous optical properties and have adequate mechanics and tunable swelling to provide an effective tamponade, restoring ocular volume and preventing retinal detachment. Conclusions to the best mechanics of vitreous humor substitutes varies, where some suggest a range of native vitreous shear storage modulus from 5 to 15 Pa are desirable. However, these values may not accurately represent the vitreous in vivo as the properties of the vitreous body changes upon removal, and the modulus of the vitreous with an intact membrane is much higher than the vitreous humor alone.

A polymer with higher mechanical integrity will better absorb shock, provide an effective tamponade, and have increased stability and retention time. For these reasons, some suggest vitreous substitutes should have shear storage moduli above from 15 to 100 Pa, or greater. In this example, a range of mechanical properties were investigated to provide a versatile biomaterial for vitreous humor substitutes. For each S/H ratio tested, 3H₂O₂ concentrations (low, medium, and high) were chosen, which directly relate to crosslinking density, allowing the biomaterial to exhibit shear storage moduli in three ranges; below 30 Pa, between 30-110 Pa, and above 110 Pa, respectively.

For the composite hydrogels, H₂O₂ concentration had a direct effect on the initial shear storage modulus. The modulus of hydrogels with no silk (0S/100H) did not change due to H₂O₂ concentration. Each ratio had different H₂O₂ concentrations independently chosen for the low, medium, and high ranges. Hydrogels formed with the same concentration of H₂O₂ (Table 1) was used to determine changes due to polymeric ratio. For the composite hydrogels, decreasing silk concentration increased the initial storage modulus. It is possible that this result could be due to multiple factors.

Decreasing the HA concentration and increasing silk concentration led to decreased mechanical strength in the composite hydrogels. However, when the silk was completely removed, the modulus decreased compared to the 10S/90H hydrogel. Without being bound to a particular theory, it is contemplated that the inclusion of the shorter silk protein in comparison to the larger HA polysaccharide may help to reduce steric hindrance and increase inter-chain crosslinking.

Ideal vitreous substitutes are stable for extended periods of times, preferably over 3 months. Enzymatically crosslinked silk hydrogels exhibit an increase in mechanics over time due to a conformational change from primarily random coil to beta sheet secondary structure with changes starting as early as 2 weeks. Increasing HA concentration in a silk-HA composite hydrogel prolonged and reduced stiffening due to beta sheet crystallinity, presumably by increasing water content and shielding the hydrophobic domains in the silk chains to reduce their self-assembly into beta sheets.

The 50S/50H sample had had a 4×, 2×, and 1.6× increase in modulus for the low, medium, and high H₂O₂ concentrations, while the other samples exhibited stable mechanics after 1 month. The larger changes in mechanics from lower H₂O₂ concentrations may be a result of lower crosslinking density and a less constrained hydrogel that better facilitates silk protein aggregation and beta sheet crystallization.

Both polymer ratio and H₂O₂ concentration affected the swelling properties of the hydrogels. Controlled swelling in vitreous humor is advantageous for maintaining intraocular pressure and for keeping the retina in place. The degree of swelling may be tuned by crosslinking density, where a decrease in crosslinking density results in a less constrained network, allowing hydrogels to swell until equilibrium is established. Lower H₂O₂ concentrations for composite hydrogels allowed for more swelling to occur.

For the 50S/50H and 25S/75H ratios, high H₂O₂ concentration provided a highly constrained network, reducing significant volumetric changes. Swelling may also be tuned using the polymeric ratio. For the 50S/50H ratios, all hydrogels equilibrated after 15 days to their original volume, even if initial swelling was observed. The 50S/50H had a significantly lower volume when compared to polymer ratios with higher HA concentrations.

It is desirable for vitreous humor substitutes to be optically clear. Optical transparency and a refractive index similar to that of native vitreous humor is desirable to provide clear vision. The refractive indexes of the hydrogels are within the range of native vitreous humor (1.334-1.337). The percentage of visible light transmitted initially and after 1 month, ranges between 75-91% depending on wavelength and time, which is a slight decrease from native vitreous at 85-95% light transmittance. All hydrogels exhibited lower transmittance at lower wavelengths, which was similar to that of native vitreous, (˜85% transmittance at 400 nm) and unlike silicone oil (100% transmittance at 400 nm). This reduced transmittance at lower wavelengths may be advantageous as it helps to prevent damage to retinal epithelium. At 1 month, the 50S/50H low and 25S/75H low showed significant differences for all visible light wavelengths compared to the saline control. These samples that have higher silk ratios along with less crosslinking density, which could have facilitated protein aggregation and crystallization causing a slight decrease in transparency.

The silk-HA composite hydrogels in the present example provide vitreous humor substitutes. Hydrogels for vitreous humor substitutes should exhibit gelation rates suitable for in situ gelation (˜10-15 minutes) to allow for injection through small gauge needles, exhibit appropriate mechanical properties (2 to >100 Pa shear storage modulus), have extended degradation rates, be biocompatible, optically clear (85-95% light transmittance, 400-700 nm), have controllable swelling, and exhibit stability over at least 6 months (time at which the clinical standard, silicone oil, should be removed to avoid toxicity).

The silk-HA composite hydrogels provided in the present example can be tuned to achieve these desired properties. In particular, the gelation times of the hydrogels can be tuned through the polymeric ratio and HRP concentration to have times between from 3 to 20 minutes, providing desirable gelation rates for in situ gelation. The silk-HA composite hydrogels have tunable mechanical properties, which may be tuned through the polymer concentration, the polymeric ratio, the H₂O₂ concentration with shear storage modulus ranging from ˜8 to 3850 Pa. The silk-HA composite hydrogels have mechanical and optical stability over time. The composite silk-HA hydrogels may be visually clear with 75-91% light transmission.

In this study we have shown that optically clear silk-HA hydrogels can be generated with a large range of mechanical and swelling properties through altering the polymeric ratio and crosslinking density with gelation time that is amenable for in situ gelation.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A biocompatible injectable composition comprising:
silk fibroin;
hyaluronic acid;
horseradish peroxidase;
hydrogen peroxide; and
water,
wherein the injectable composition undergoes gelation from a non-gelated solution to a hydrogel upon mixing of the silk fibroin, hyaluronic acid, horseradish peroxidase, hydrogen peroxide, and water,
wherein a polymeric ratio of silk fibroin and hyaluronic acid and a horseradish peroxidase concentration are adapted to provide a gelation time of between 3 minutes and 20 minutes inclusive,
wherein a silk fibroin concentration, a hyaluronic acid concentration, the polymeric ratio, and a hydrogen peroxide concentration are adapted to provide a storage modulus of between 6 Pa and 4000 Pa inclusive,
wherein the injectable composition is adapted to provide a viscosity of the non-gelated solution that is adequately low to allow injection via a needle having a size of between 32 gauge and 18 gauge inclusive,
wherein the injectable composition is adapted to provide an optical transmittance of the hydrogel of between 75% and 95%, inclusive, for at least one wavelength of between 400 nm and 700 nm inclusive,
wherein the injectable composition is adapted to provide a hydrogel swellability of the hydrogel including a volume expansion from 5% to 400%, inclusive, relative to an original volume of the hydrogel after soaking in an aqueous solution for 12 hours,
wherein the injectable composition is adapted to provide a hydrogel stability by maintaining at least 75% of the storage modulus and the optical transmittance of the hydrogel after 6 months in vivo.

2. A method of forming a biocompatible injectable composition, the method comprising:
contacting silk fibroin, hyaluronic acid, horseradish peroxidase, hydrogen peroxide, and water in a solution in amounts sufficient such that the solution undergoes gelation to a hydrogel,
wherein a polymeric ratio of the silk fibroin and the hyaluronic acid and the horseradish peroxidase concentration are adapted in the solution to provide a gelation time of between 3 minutes and 20 minutes inclusive,
wherein a silk fibroin concentration, a hyaluronic acid concentration, the polymeric ratio, and a hydrogen peroxide concentration are adapted to provide a storage modulus of between 6 Pa and 4000 Pa inclusive,
wherein the injectable composition is adapted to provide a viscosity of the non-gelated solution that is adequately low allow injection via a needle having a size of between 32 gauge and 18 gauge inclusive,
wherein the injectable composition is adapted to provide an optical transmittance of the hydrogel of between 75% and 95%, inclusive, for at least one wavelength of between 400 nm and 700 nm inclusive,
wherein the injectable composition is adapted to provide a hydrogel swellability of the hydrogel including a volume expansion from 5% to 400%, inclusive, relative to an original volume of the hydrogel after soaking in an aqueous solution for 12 hours,
wherein the injectable composition is adapted to provide a hydrogel stability by maintaining at least 75% of the storage modulus and the optical transmittance of the hydrogel after 6 months in vivo.

3. A kit comprising components for a biocompatible injectable composition, the kit comprising:
silk fibroin;
hyaluronic acid;
horseradish peroxidase;
hydrogen peroxide; and
water,
wherein the silk fibroin, the hyaluronic acid, the horseradish peroxidase, the hydrogen peroxide, and the water are separated to prevent gelation,
wherein when the components in the kit are combined the injectable composition undergoes gelation from a non-gelated solution to a hydrogel upon mixing of the silk fibroin, hyaluronic acid, horseradish peroxidase, hydrogen peroxide, and water,
wherein a polymeric ratio of silk fibroin and hyaluronic acid and a horseradish peroxidase concentration are adapted in the hydrogel to provide a gelation time of between 3 minutes and 20 minutes inclusive,
wherein a silk fibroin concentration, a hyaluronic acid concentration, the polymeric ratio, and a hydrogen peroxide concentration are adapted in the hydrogel to provide a storage modulus of between 6 Pa and 4000 Pa inclusive,
wherein the injectable composition is adapted in the hydrogel to provide a viscosity of the non-gelated solution that is adequately low to allow injection via a needle having a size of between 32 gauge and 18 gauge inclusive,
wherein the injectable composition is adapted in the hydrogel to provide an optical transmittance of the hydrogel of between 75% and 95%, inclusive, for at least one wavelength of between 400 nm and 700 nm inclusive,
wherein the injectable composition is adapted in the hydrogel to provide a hydrogel swellability of the hydrogel including a volume expansion from 5% to 400%, inclusive, relative to an original volume of the hydrogel after soaking in an aqueous solution for 12 hours,
wherein the injectable composition is adapted in the hydrogel to provide a hydrogel stability by maintaining at least 75% of the storage modulus and the optical transmittance of the hydrogel after 6 months in vivo.

4. The kit of claim 3, wherein the silk fibroin and the hyaluronic acid are separated from the horseradish peroxidase in the kit.

5. The biocompatible injectable composition, the method, or the kit according to any one of the preceding claims, wherein the injectable composition consists essentially of the silk fibroin, the hyaluronic acid, the horseradish peroxidase, the hydrogen peroxide, and the water.

6. The biocompatible injectable composition, the method, or the kit according to any one of the preceding claims, wherein the injectable composition consists of the silk fibroin, the hyaluronic acid, the horseradish peroxidase, the hydrogen peroxide, and the water.

7. The biocompatible injectable composition, the method, or the kit according to any one of the preceding claims, wherein the silk fibroin and the hyaluronic acid form a total polymer content, and wherein the total polymer content in the composition ranges from 0.1% (w/v) to 10% (w/v) inclusive.

8. The biocompatible injectable composition, the method, or the kit according to any one of the preceding claims, wherein the silk fibroin is present at a concentration from 0.05% (w/v) to 9.9% (w/v) inclusive.

9. The biocompatible injectable composition, the method, or the kit according to any one of the preceding claims, wherein the hyaluronic acid is present at a concentration from 0.05% (w/v) to 9.9% (w/v) inclusive.

10. The biocompatible injectable composition, the method, or the kit according to any one of the preceding claims, wherein the silk fibroin and the hyaluronic acid are present in a ratio of 5:95 to 95:5 inclusive (silk fibroin:hyaluronic acid).

11. The biocompatible injectable composition, the method, or the kit according to any one of the preceding claims, wherein the injectable composition is adapted to provide an optical transmittance of the hydrogel of between 75% and 95%, inclusive, for all wavelengths between 400 nm and 700 nm inclusive.

12. The biocompatible injectable composition, the method, or the kit according to any one of the preceding claims, wherein the injectable composition is adapted to provide a viscosity of the non-gelated solution that is adequately low to allow injection via a needle having a size between 32 gauge and 28 gauge inclusive.

13. The biocompatible injectable composition, the method, or the kit according to any one of the preceding claims, wherein the injectable composition includes the horseradish peroxidase at a concentration from 0.01 U/mL to 10 U/mL.

14. The biocompatible injectable composition, the method, or the kit according to any one of the preceding claims, wherein the injectable composition includes the hydrogen peroxide at a concentration from 0.001% volume by volume (v/v) to 1% (v/v), inclusive, based on the total volume of the injectable composition.

15. The biocompatible injectable composition, the method, or the kit according to any one of the preceding claims, wherein the polymeric ratio of silk fibroin and hyaluronic acid and a horseradish peroxidase concentration are adapted to provide a gelation time of between 10 minutes and 15 minutes inclusive.

16. The biocompatible injectable composition, the method, or the kit according to any one of the preceding claims, wherein the injectable composition is adapted to provide an optical transmittance of the hydrogel of between 85% and 95%, inclusive, for at least one wavelength of between 400 nm and 700 nm inclusive.

17. The biocompatible injectable composition, the method, or the kit according to any one of the preceding claims, wherein a silk fibroin concentration, a hyaluronic acid concentration, the polymeric ratio, and a hydrogen peroxide concentration are adapted to provide a storage modulus of between 6 Pa and 100 Pa inclusive.

18. The biocompatible injectable composition, the method, or the kit according to any one of the preceding claims, wherein a silk fibroin concentration, a hyaluronic acid concentration, the polymeric ratio, and a hydrogen peroxide concentration are adapted to provide a storage modulus of between 100 Pa and 4000 Pa inclusive.

19. The biocompatible injectable composition, the method, or the kit according to any one of the preceding claims, wherein a silk fibroin concentration, a hyaluronic acid concentration, the polymeric ratio, and a hydrogen peroxide concentration are adapted to maintain the swellability values within the specified ranges for a duration, wherein the duration is at least one month.

20. The biocompatible injectable composition, the method, or the kit according to any one of the preceding claims further comprising an additive.
